(19) United States
(12) Patent Application Publication
(10) Pub. No.: US 2016/0038309 A1
(43) Pub. Date: Feb. 11, 2016

(54) SPINAL FUSION APPARATUS AND RELATED METHODS

(71) Applicant: Michael J. DOYLE, Louisville, KY (US)

(72) Inventor: Michael J. DOYLE, Louisville, KY (US)

(21) Appl. No.: 14/922,965

(22) Filed: Oct. 26, 2015

Related U.S. Application Data
(63) Continuation-in-part of application No. PCT/US2014/035297, filed on Apr. 24, 2014.
(60) Provisional application No. 61/815,383, filed on Apr. 24, 2013.

(51) Int. Cl. A61F 2/46; A61B 17/17

(52) U.S. Cl. CPC A61F 2/4611; A61B 17/1757

(57) ABSTRACT

An apparatus allows for posterior lumbar interbody fusion to be performed with a stable construct to achieve arthrodesis. A fastener is adapted for connecting the superior vertebra to the inferior vertebra. A receiver for positioning between the superior and inferior vertebrae includes a passage for receiving the fastener. The receiver comprises a first surface for positioning adjacent the first vertebrae and a second surface for positioning adjacent to the inferior vertebrae, and the passage extends at an angle relative to the first and second surfaces. Related tools and methods are also described.

The disclosure of international patent application PCT/US 14/35397 is incorporated herein by reference.

### TECHNICAL FIELD

This disclosure relates generally to spinal fusion, and more particularly, to an interbody fusion apparatus, related tool and methods of use.

### BACKGROUND OF THE INVENTION

Lumbar interbody fusion in which spacers are placed within the disc space along with bone graft has become a widely used and preferred means of achieving lumbar arthrodesis and is presently used to treat a variety of lumbar pathologies. Once the spacers have been placed in the disc space, pedicle screws are frequently utilized to provide additional lumbar stabilization and also to secure the interbody spacers and prevent posterior migration by compressing across the disc space.

Unfortunately, placement of pedicle screws is fraught with a variety of potential complications. The screw and rod construct is placed within the paraspinous muscles and frequently causes damage to the muscle, fibrosis and potentially chronic pain. In addition, placement of pedicle screws potentially denervates the paraspinous muscles and may potentially destabilize adjacent spinal segments, possibly making it more likely that the patient will develop adjacent level degenerative disease and require extension of the fusion to adjacent levels. Finally, placement of the pedicle screws requires a trajectory and entry point and screw trajectory which passes close to and unfortunately often through the adjacent level facet joints, often damaging the facet joints and promoting adjacent level degenerative disease.

As an alternative to posterior interbody fusion with pedicle screw fixation, anterior interbody spacers have been developed which have an integrated screw system that engages the vertebral body, fixes the spacer in position and partially stabilizes the spinal segment to facilitate fusion. Unfortunately, the available integrated spacer screw constructs are placed from an anterior approach. Most spinal surgeons are not as comfortable with an anterior approach because such an approach requires mobilization of large blood vessels and potentially disastrous injury to the large blood vessels in addition to other well-known complications unique to the anterior approach. In addition, the anterior approach does not allow the thorough decompression of facet and ligamentous hypertrophy which are common sources of stenosis and nerve root impingement and also does not require as extensive decompression of spinal canal and nerve roots via a posterior discectomy.

Accordingly, a need is identified for an improved spinal fusion technique and related apparatus.

### SUMMARY

According to a first aspect of the disclosure, an apparatus for connecting superior and inferior vertebrae is provided. The apparatus comprises a fastener adapted for connecting the superior vertebra to the inferior vertebra, and a receiver for positioning between the superior and inferior vertebrae. The receiver includes a passage for receiving the fastener, the receiver having a first surface for positioning adjacent the first vertebrae and a second surface for positioning adjacent to the inferior vertebrae, the passage extending at an angle relative to the first and second surfaces.

In one embodiment, the receiver includes first and second passages, each adapted for receiving a fastener for connecting with the superior and inferior vertebrae. The first and second passages may extend at angles relative to each other.

In this or any other embodiment, a tool may be adapted for temporarily connecting with the receiver. The tool may include a guide for guiding a drilling element for forming an opening in one of the superior or inferior vertebrae when connected to the receiver. The guide may be tubular, and may be connected to the tool for movement along an arcuate path to a position in alignment with the angled passage of the receiver, or may be fixed in alignment with the angled passage of the receiver. The tool may also comprise a connector adapted for connecting to the body of the receiver.

A further aspect of the disclosure includes an apparatus for use in connection with a procedure for connecting superior and inferior vertebrae using a receiver including at least one passage. The apparatus comprises a tool adapted to be connected to the receiver for guiding a drilling element for forming an opening in one of the superior or inferior vertebrae. The tool may comprise a guide adapted for receiving the drilling element. The guide may be connected to the tool for movement along an arcuate path to a position in alignment with the angled passage of the receiver. The tool may include a connector for connecting to the receiver in a manner that prevents relative rotation during use in forming the opening on one of the superior or inferior vertebrae, and also a manner that allows relative rotation after forming the opening on one vertebrae to form an opening in the other vertebra.

A further aspect of the disclosure pertains to an apparatus for use in connection with a procedure for connecting superior and inferior vertebrae. The apparatus comprises a receiver for positioning between the superior and inferior vertebrae. The receiver is adapted for positioning between the superior vertebra and the inferior vertebra. A tool is adapted to be connected to the receiver for guiding a drilling element for forming an opening in one of the superior or inferior vertebrae.

In one embodiment, the receiver includes at least one passage adapted for receiving a fastener. The passage extends at an angle relative to a first surface of the receiver for positioning adjacent to the superior vertebra and a second surface of the receiver for positioning adjacent to the inferior vertebra. The tool may comprise a guide adapted for receiving the drilling element. The guide may be connected to the tool for movement along an arcuate path to a position in alignment with the angled passage of the receiver. The tool may include a connector for connecting to the receiver in a manner that prevents relative rotation during use in forming the opening on one of the superior or inferior vertebrae, and also in a manner that allows relative rotation after forming the opening on one vertebrae to form an opening in the other vertebra.

### BRIEF DESCRIPTION OF THE DRAWING FIGURES

FIG. 1 is a side view of one embodiment of a device according to the disclosure;

FIG. 2 is a top view of one embodiment of the device of FIG. 1;

FIGS. 3A, 3B, 3C and 4 are side schematic side views of the device of FIG. 1 in use;

FIG. 5 is a side schematic view of a tool for use in connection with the device;

FIG. 6 is a partially cutaway plan view of the tool of FIG. 5;

FIG. 7 is a side schematic view of the tool of FIG. 5 in use;

FIGS. 8A, 8B, and 8C are partially cutaway side views of drilling elements for use in connection with the device; and

FIGS. 9, 10 and 11 illustrate an alternate embodiment of the tool.

### MODES FOR CARRYING OUT THE INVENTION

The description provided below and in regard to the figures applies to all embodiments unless noted otherwise.

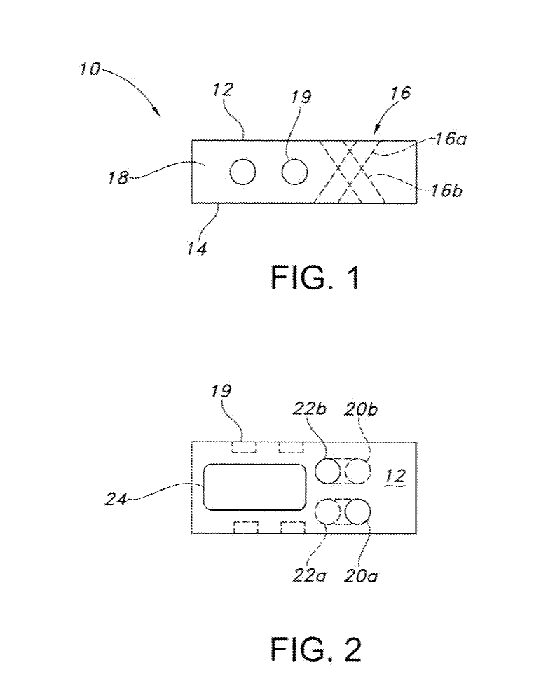

With specific reference to FIG. 1, a device 10 for receiving fasteners, and thus termed a receiver, is illustrated for insertion between adjacent vertebrae in association with spinal fusion. The device 10 may include an upper surface 12 and a lower surface 14, and may further include at least one lateral or side surface 18. In one embodiment, the upper 12 and lower surfaces 14 may be substantially parallel to one another and, in any case, are adapted for being positioned adjacent corresponding surfaces of superior and inferior vertebrae, which are termed endplates. As will be understood in more detail upon reviewing the description that follows, the device 10 may include one or more surface formations 19 for aiding in fixing the device in position. These surface formations 19 may be positioned on the lateral surface 18, and may be in the form of one or more recesses.

At least one passage 16 may traverse the device 10, such as at an oblique angle to one or more surfaces of the device 10. In one embodiment, the device 10 includes a first passage 16a and a second passage 16b. Each passage 16A, 16B may extend between the upper surface 12 and the lower surface 14 at an oblique angle. The passages 16a, 16b may include internal threads for receiving a screw or other threaded device, and may include a continuous sidewall (but this is considered optional).

As illustrated in FIGS. 1 and 2, the first passage 16a may include an inlet 20a on the upper surface 12, and an outlet 22a on the lower surface 14. The second passage may include an inlet 20b on the lower surface 14 and an outlet 22b on the upper surface 12. In use, the inlet 20a of the first passage 16a is proximal of the inlet 22b of the second passage 16b, and likewise, the outlet 20b of the second passage 16b is proximal of the outlet 22a of the first passage 16a.

In one embodiment, the device 10 may further provide a space for bone graft material to more securely fix the adjacent vertebrae during fusion. This may be accomplished using a holder 24 that extends at least partially into or through the device 10. More than one holder may be provided, if desired, and the shape and size of the holder may be varied from what is illustrated, depending on the particular application.

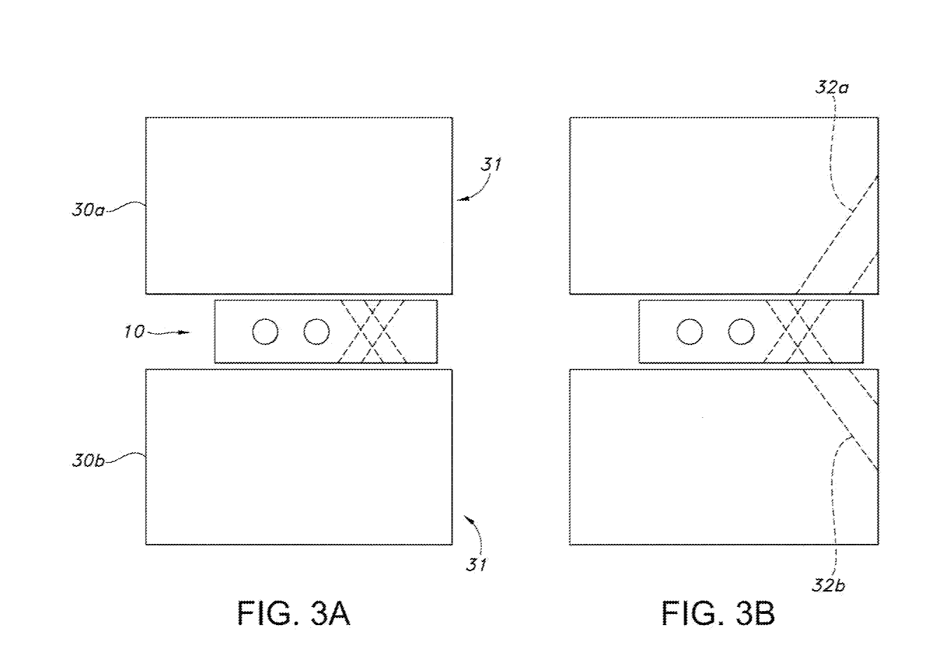

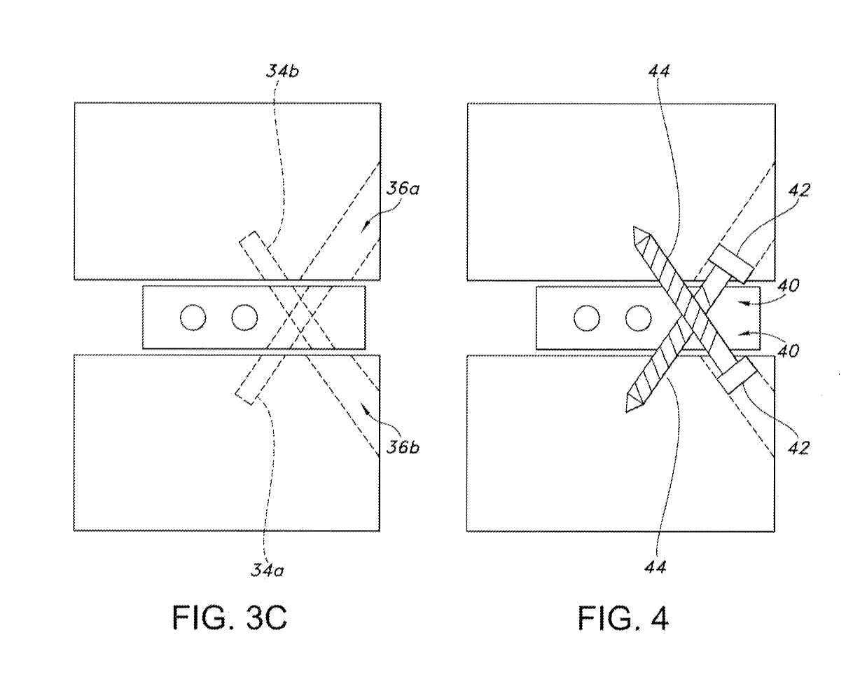

FIGS. 3a-3c illustrate an example of one possible use of the device 10 in practice. As shown in FIG. 3a, the device may be inserted between two adjacent vertebrae 30a, 30b, such as between the superior and inferior endplates. Upon insertion, the portion of the device 10 including the first and second passages 16a, 16b may align with a posterior portion 31 of the vertebrae 30a, 30b, such as a posterior portion of the vertebral body. In the case of a PLIF, the passages 16a, 16b may be placed at a medial location of the posterior vertebral body. In the case of a transforaminal lumbar interbody fusion (TLIF), the passages 16a, 16b may be placed at a more lateral location of the posterior vertebral body.

As shown in FIG. 3b, first boreholes 32a, 32b may be formed in the adjacent vertebrae 30a, 30b. These first boreholes 32a, 32b may align with the first and second passages 16a, 16b of the device 10. For example, the first borehole 32a in the superior vertebra 30a may align with the first passage 16a of the device. Similarly, the first borehole 32b in the inferior vertebra 30b may align with the second passage 16b of the device. These first boreholes 32a, 32b may be larger in diameter than the first and second passages 16a, 16b of the device 10.

FIG. 3c illustrates the further formation of second boreholes 34a, 34b in the adjacent vertebrae 30a, 30b. These second boreholes 34a, 34b may align with the first boreholes 32a, 32b, respectively, and the first and second passages 16a, 16b of the device, but may extend to an adjacent vertebra. These second boreholes 34a, 34b may be smaller in diameter than the first boreholes 32a, 32b. As illustrated, second borehole 34a may be positioned in the inferior vertebra 30b, while being aligned with the first borehole 32a in the superior vertebra 30a, as well as the first passage 16a of the device 10.

This combination of the first borehole 32a in the superior vertebra 30a, the first passage 16a, and the second borehole 34a in the inferior vertebra 30b, may form a first interveterbral passage 36a, descending from the superior vertebra 30a, through the device 10, and into the inferior vertebra 30b. Similarly, second borehole 34b in the superior vertebra 30a may align with the first borehole 32b in the inferior vertebra 30b and the second passage 16b to form a second intervertebral passage 36b ascending from the inferior vertebra 30b, through the device 10, and into the superior vertebra 30a.

With reference to FIG. 4, upon completion of the intervertebral passages 36a, 36b, one or more fasteners, such as screws 40 (which may be lag screws, having a shank including a threaded distal end and a smooth proximal portion adjacent to the head), may be used to fix the vertebrae 30a, 30b in relative position to one another. For example, the screws 40 may enter through the first boreholes 32a, 32b, and at least partially pass through the passages 16a, 16b of the device 10, and into the second boreholes 34a, 34b, as shown in FIG. 4. The dual action of a first screw 40 anchoring the adjacent vertebrae 30a, 30b from a superior position and a second screw anchoring the adjacent vertebrae 30a, 30b from an inferior position serves to create a more secure fusion.

In one embodiment, as shown in FIG. 4, a first portion such as the head 42 of a first screw 40 may remain in a superior vertebra 30a, while a second portion such as the tail 44 of the screw 40 may pass through the device 10 and into the inferior vertebra 30b. Similarly, the head 42 of a second screw 40 may remain in the inferior vertebra 30b, while the tail 44 may pass through the device 10 and into the superior vertebra 30a. The heads 42 may also be adapted to expand so as to provide a locking function.

In another embodiment, the head of each screw 40 may pass completely through the first borehole 32a, 32b, and may be secured in or on the device 10, allowing the tail 44 to pass into an adjacent vertebra 30a, 30b. The screw 40 may include an expandable portion, such as the head, or engage an expandable nut associated with the device 10, in order to prevent migration or backout. Thus, each screw 40 may individually secure a single vertebra to the device 10 without securing the adjacent vertebra to the device. In this embodiment, the dual action of the first screw 40 securing the inferior vertebra 30b to the device 10 and the second screw 40 securing the superior vertebra 30a to the device 10 serves to prevent relative motion of either vertebra 30a, 30b with respect to the device 10.

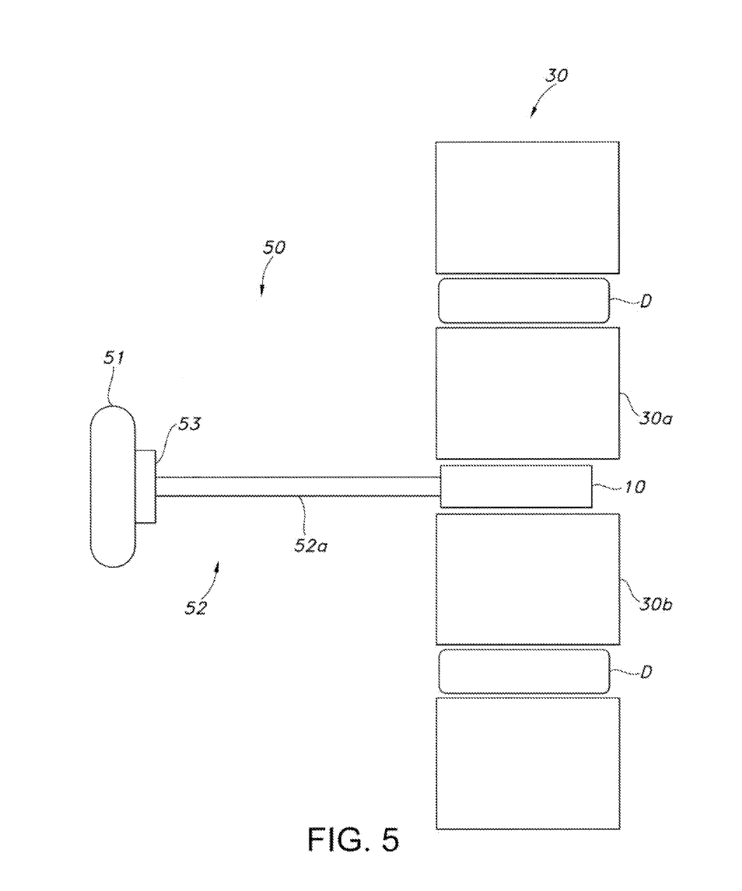

With reference to FIG. 5, the insertion of the device 10 and/or the drilling and insertion of screws 40 may be aided by the use of tool, such as an inserter 52, for placing the device 10 in between the two adjacent vertebrae 30a, 30b in the vertebral column 30. The device 10 may replace a disc D normally between vertebrae, or may be placed between two adjacent vertebrae 30a, 30b in combination with at least a portion of a compromised disc.

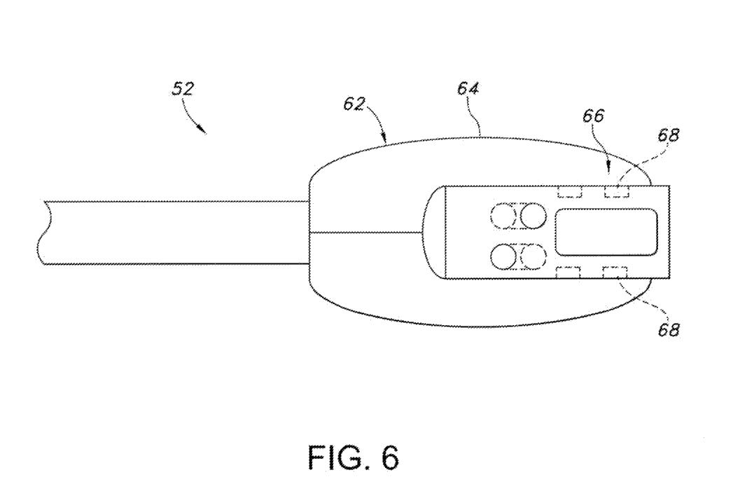

As illustrated in FIG. 6, the inserter 52 may be used to grip the device 10 for insertion. For example, a retainer 62 such as lockable clamp 64 with releasable jaws may be provided at the end of a shaft 52a for engaging the device 10 for insertion, and then being released later. In one embodiment, the clamp 64 may include one or more formations 66 for engaging the surface formations 19 on the device 10, which again may comprise recesses. These formations 66 may be in the form of projections 68 for insertion into the recesses of the device 10. The interaction between the engagement formations 66 on the inserter 52 and the surface formations 19 on the device 10 may serve to limit or prevent relative motion of the device 10 within the retainer 62.

In further reference to FIG. 5, the inserter 52 may be adapted for attachment to various components. For example, the inserter 52 may be provided with a releasable connector 53 for connecting with an implement 51 adapted for being manually grasped by the clinician or struck with another implement (e.g., a mallet) or the like during the process of installing the device 10. The connector 53 may comprise, for example, a bayonet style connection or a threaded connection, and may include a removable pin for preventing relative rotation between the implement and the device 52 when connected.

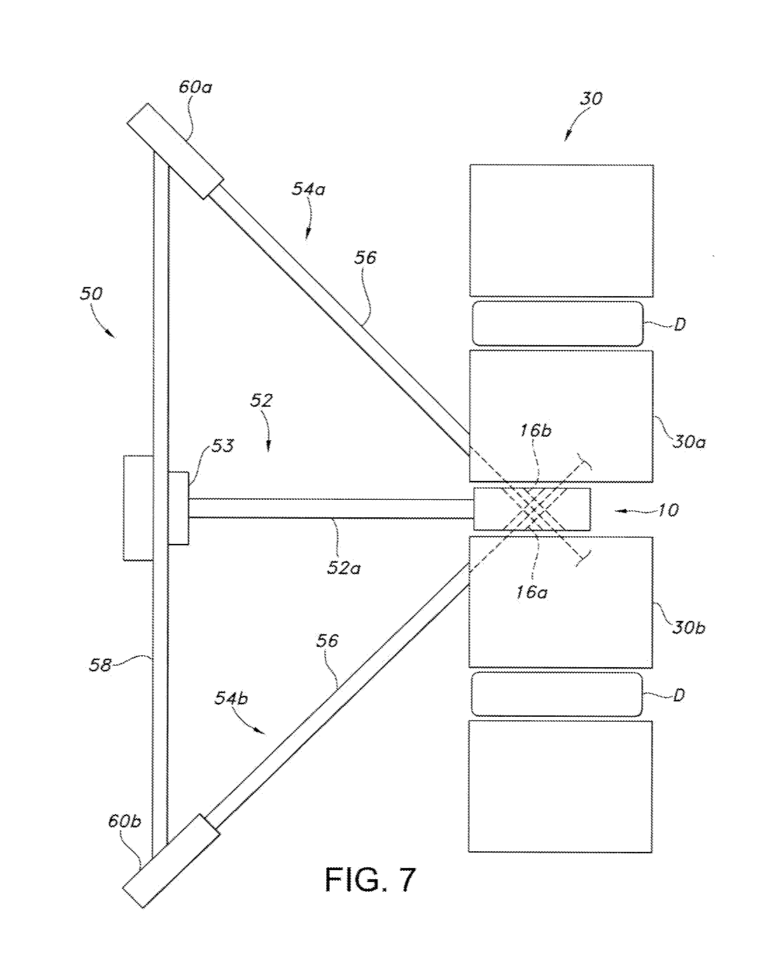

Turning now to FIG. 7, the installation tool 50 may be adapted to include first and second guides 54a, 54b for assisting in drilling the vertebrae 30a, 30b, and installing the screws 40. These guides 54a, 54b may comprise hollow tubes 56, which may be rigid. Alternately, guides 54a, 54b may include any structure capable of providing a fixed trajectory for a tool, such as a track, a shaft, or the like. In practice, the tool guides 54a, 54b may be used to guide a tool such as a drill to a specified location on a subject, such as a particular entry point on a vertebra to be drilled. The ends of the tubes 56 for positioning adjacent to the vertebrae may be chamfered, as illustrated, so as to allow for flush contact to be made. The guides 54a, 54b may be provided in different sizes (e.g., lengths) for use with patients having different anatomies (e.g., a particularly large subject may require a longer guide).

The tool guides 54a, 54b may be attached to the installer 50 by way of first and second receivers 60a, 60b, as shown in FIG. 7. These receivers 60a, 60b may be fixed, or may comprise a hinge element such as a compound locking or hinge or like mechanism, such as a ratchet, for accurately setting and fixing a position of the tool guide 54a, 54b. The receivers 60a, 60b may be adjusted in any plane so as to coordinate with the anatomy of the subject, such that the tool guides 54a, 54b may align with a desired entry point on the vertebrae 30a, 30b.

A connector 58 may be provided to join the receivers 60a, 60b. Thus, the receivers 60a, 60b may have a relatively fixed point of reference from which to adjust an angle of the tool guides 54a, 54b. The inserter 52 may also be fixed to the connector 58 of the installation tool 50 through the releasable connector 53, which again may be arranged to prevent relative rotation in the locked condition. In one embodiment, inserter 52 may be extendable and retractable, such that a distance between the device 10 and the connector 58 may be adjusted and maintained. Accordingly, the tool guides 54a, 54b and the inserter 52 may be maintained in relatively fixed positions during the procedure.

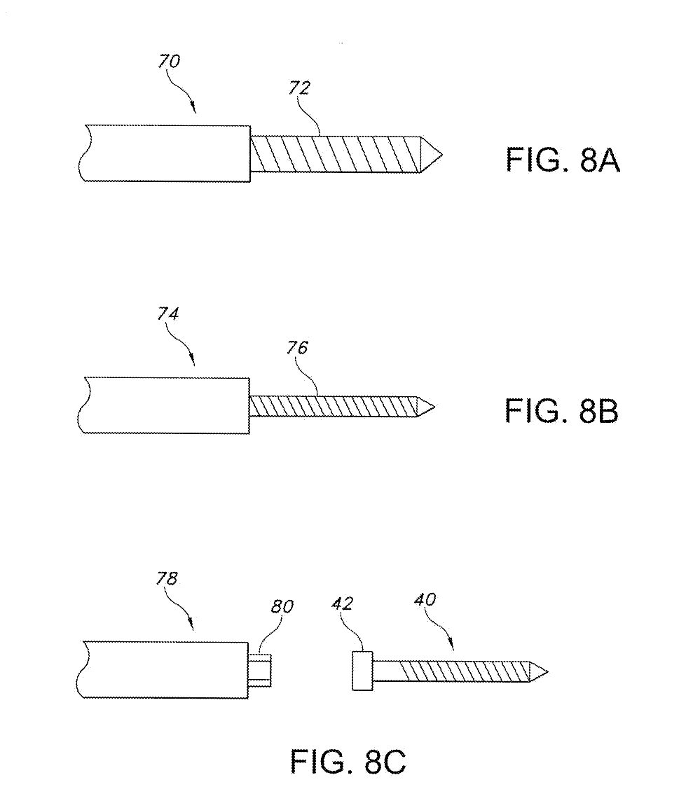

With reference to FIGS. 8a-8c, various tools are disclosed that may be used in combination with the installation tool for fusing the two adjacent vertebrae 30a, 30b. In use, the first and second receivers 60a, 60b may be set at the desired angle for the fusion procedure. A first drilling element 70, including a first drill bit 72 may be passed along the first tool guide 54a, such as through tube 56, to the desired entry point on the superior vertebra 30a. The first drill bit 72 may then be used to drill the first borehole 32a at an oblique angle. The first drilling element 70 may be removed and replaced with a second drilling element 74, including a second drill bit 76. This second drill bit may be passed along the first tool guide 54a, through the first borehole 32a, through the first passage 16a, and into the inferior vertebra 30b to form second borehole 34a. The first drill bit 72 may be larger in diameter than the second drill bit 76.

As can be appreciated, the fixed angle of the first receiver 60a ensures that the angle of entry of the first and second boreholes is substantially identical. The combination of the use of the first drilling element 70 and the second drilling element 74 along the fixed angle results in the creation of the first intervertebral passage 36a. Similarly, the second tool guide 54b may be used to form the second intervertebral passage 36b through the use of the first and second drilling elements 70, 74 being passed along the second tool guide 54b to create the first borehole 32b in the inferior vertebra 30b and the second borehole 34b in the superior vertebra 30a, respectively. The creation of the intervertebral passages 36a, 36b may be accomplished simultaneously, or sequentially.

A driver 78 may be used to install the screws 40 within the intervertebral passages 36a, 36b. The driver 78 may include a driving head 80 for interacting with a head 42 of a screw 40 in order to drive the screw 40 into a desired position. The driver 78 (and screw 40) may be passed along the tool guides 54a, 54b in order to ensure that the angle of entry of the screws 40 corresponds to the oblique angles of the intervertebral passages 36a, 36b. The screws 40 may be tightened to a desired depth to ensure that the device is sufficiently secured in position between the vertebrae 30a, 30b, and to ensure that the vertebrae 30a, 30b are fixed relative to one another. The driver 78 may also be adapted for driving the drills 70, 72.

Upon securing the device 10 between the adjacent vertebrae 30a, 30b through the use of the screws 40, the inserter 52 may release the device 10, and the installation tool 50 may be removed. Corresponding procedures can then be taken to pack the site with bone material or the like.

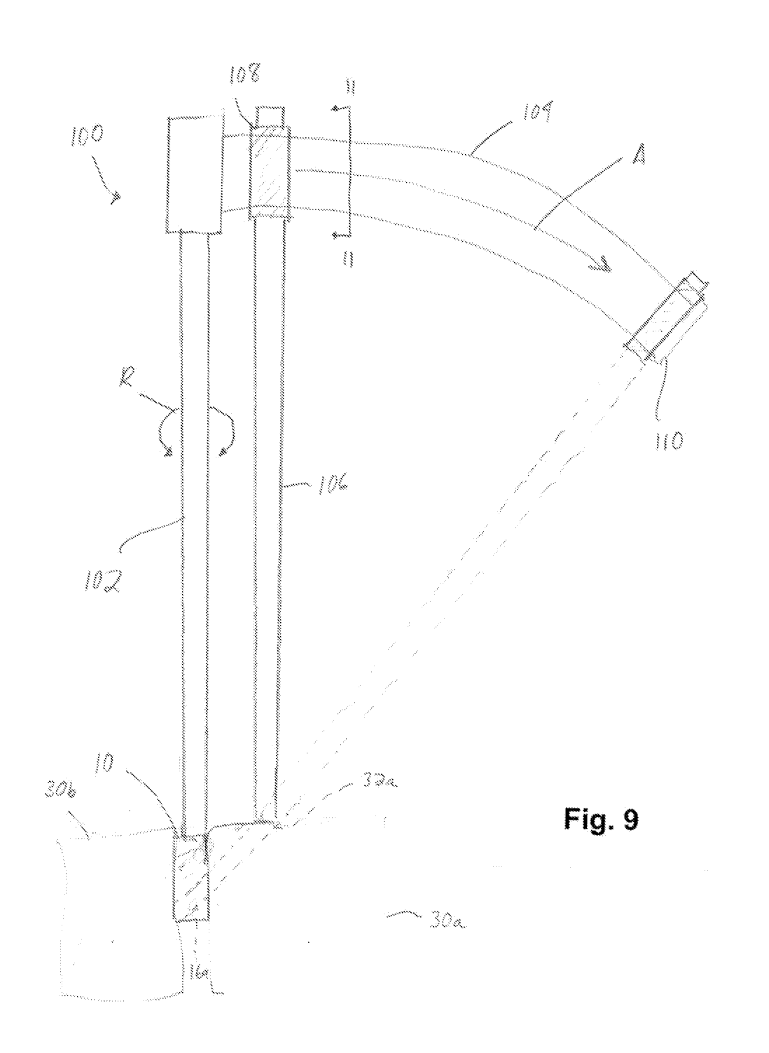

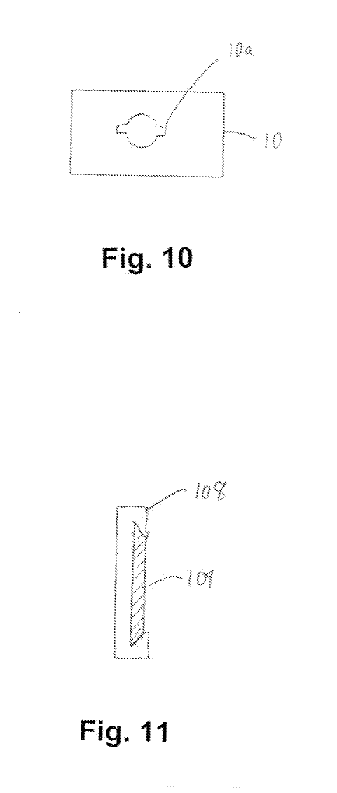

A further embodiment of an installation tool 100 is shown in FIG. 9. In this embodiment, the tool 100 includes a shaft 102 for connecting with the device 10 at one end, such as by threaded connection as noted above, but possibly also using a keyway 10a in the device for engaging a portion of the tool 100 to prevent relative rotation (see FIG. 10). An opposite end portion of the shaft 102 includes a generally arcuate guide 104 to which a tool guide 106 is attached, such as to allow for relative sliding movement along an arcuate path A. The attachment may be made between the guide 104 by a dovetail type arrangement with a connector 108 on the tool guide (see FIG. 11) to allow for free sliding movement. Regardless of the particular form of attachment, the connection should allow for movement of the tool guide 106 between a condition in which the tool guide is generally aligned with the shaft 102 and in contact with a target point on the vertebra (which may actually be closer to the shaft than the spacing shown, which is done only for purposes of illustration), to a position at which the tool guide is generally angled relative to the shaft and aligned with one or more openings in the device 10 (which may be controlled by a stop 110) corresponding to passages 16a, 16b. Proper alignment among the tool and the passages 16a, 16b is thus assured.

The tool guide 106, which is hollow, may then receive the drilling element for drilling into the vertebra 30a to allow for the installation of a fastener or screw through it and into device 10, as outlined in the foregoing discussion. As can be appreciated, the tool 100 (or guide 104 in particular) can simply be rotated (note arrow R) or adjusted to allow for a similar procedure to be performed on an adjacent vertebra 30b

As can now be understood, the drill/screw guide allows the drilling elements to enter the posterior cortex of the vertebral body and traverse the vertebral body obliquely and subcontact with the spacer is achieved. A smaller drilling element is then inserted along the same trajectory and passes through the spacer and drills across the endplate and into the adjacent vertebral body at an oblique angle. Following drilling along the fixed trajectory, the screw can be placed through the guide where it passes through the spacer, engages the adjacent vertebral body and eventually locks into the spacer securely to provide for a very secure construct. The drill/screw guide allows for placement of two screws through each spacer, one that enters from above to secure the vertebral body and one entering from below to secure the above vertebral body.

One advantage of this procedure is that it allows for transforaminal lumbar interbody fusion, or TLIF, to be successfully performed through a relatively small and even minimally invasive incision. The posterior approach allows for excellent decompression of the canal and neural foramina and nerve roots. The ability of the spacer to securely accommodate screws allows for a very stable arthrodesis construct to be achieved through a much less invasive approach than with pedicle screw fixation. In addition, since the inserter used to place the interbody spacers incorporates the guide that allows for drilling and placement of the screws, a posterior lumbar interbody fusion (PLIF) or TLIF can be performed much more rapidly than with the use of pedicle screws for fixation. The use of this novel integrated screw construct for PLIF or TLIF has the potential to dramatically advance the patient care and dramatically enhance the results of lumbar spine surgery.

The device 10 may comprise titanium, carbon, polymers, such as PEEK, or any other material compatible with arthrodesis. Other components may be made from similar materials, and may be made for repeated or single use.

While the disclosure presents certain embodiments to illustrate the inventive concepts, numerous modifications, alterations, and changes to the described embodiments are possible without departing from the sphere and scope of the present invention. In particular the diameter and depth of each borehole may be adjusted according to the anatomy of the subject, the desired screw dimension and the desired degree of anchoring between adjacent vertebrae. Also, the drawings, while illustrating the inventive concepts, are not to scale, and should not be limited to any particular sizes or dimensions. Accordingly, it is intended that the present disclosure not be limited to the described embodiments, but that it has the full scope defined by the language of the following claims, equivalents thereof, and any modifications within the scope of the knowledge of the skilled artisan. The disclosure of U.S. Pat. No. 7,942,903 is incorporated herein by reference.

1. An apparatus for connecting superior and inferior vertebrae, comprising:
a fastener adapted for connecting the superior vertebra to the inferior vertebra; and
a receiver for positioning between the superior and inferior vertebrae, the receiver including a passage for receiving the fastener, the receiver having a first surface for positioning adjacent the first vertebrae and a second surface for positioning adjacent to the inferior vertebrae, the passage extending at an angle relative to the first and second surfaces.

2. The apparatus of claim 1, wherein the receiver includes first and second passages, each adapted for receiving a fastener for connecting with the superior and inferior vertebrae.

3. The apparatus of claim 2, wherein the first and second passages extend at angles relative to each other.

4. The apparatus of claim 1, further including a tool adapted for temporarily connecting with the receiver, the tool including a guide for guiding a drilling element for forming an opening in one of the superior or inferior vertebrae when connected to the receiver.

5. The apparatus of claim 4, wherein the guide is tubular.

6. The apparatus of claim 4, wherein the guide is connected to the tool for movement along an arcuate path to a position in alignment with the angled passage of the receiver.

7. The apparatus of claim 4, wherein the tool comprises a connector adapted for connecting to the body of the receiver.

8. The apparatus of claim 1, further including a tubular guide for guiding a drilling element for forming an opening in one of the superior or inferior vertebrae.

9. The apparatus of claim 8, wherein the tubular guide is connected to the receiver in alignment with the angled passage.

10. An apparatus for use in connection with a procedure for connecting superior and inferior vertebrae using a receiver including at least one passage, comprising:
a tool adapted to be connected to the receiver for guiding a drilling element for forming an opening in one of the superior or inferior vertebrae.

11. The apparatus of claim 10, wherein the tool comprises a guide adapted for receiving the drilling element.

12. The apparatus of claim 11, wherein the guide is connected to the tool for movement along an arcuate path to a position in alignment with the angled passage of the receiver.

13. The apparatus of claim 10, wherein the tool includes a connector for connecting to the receiver in a manner that prevents relative rotation during use in forming the opening on one of the superior or inferior vertebrae.

14. The apparatus of claim 10, wherein the tool includes a connector for connecting to the receiver in a manner that allows relative rotation after forming the opening on one vertebrae to form an opening in the other vertebra.

15. An apparatus for use in connection with a procedure for connecting superior and inferior vertebrae, comprising:
a receiver for positioning between the superior and inferior vertebrae, the receiver adapted for positioning between the superior vertebra and the inferior vertebra; and
a tool adapted to be connected to the receiver for guiding a drilling element for forming an opening in one of the superior or inferior vertebrae.

16. The apparatus of claim 15, wherein the receiver includes at least one passage adapted for receiving a fastener, the passage extending at an angle relative to a first surface of the receiver for positioning adjacent to the superior vertebra and a second surface of the receiver for positioning adjacent to the inferior vertebra.

17. The apparatus of claim 15, wherein the tool comprises a guide adapted for receiving the drilling element.

18. The apparatus of claim 17, wherein the guide is connected to the tool for movement along an arcuate path to a position in alignment with the angled passage of the receiver.

19. The apparatus of claim 15, wherein the tool includes a connector for connecting to the receiver in a manner that prevents relative rotation during use in forming the opening on one of the superior or inferior vertebrae.

20. The apparatus of claim 15, wherein the tool includes a connector for connecting to the receiver in a manner that allows relative rotation after forming the opening on one vertebrae to form an opening in the other vertebra.
